(19) United States
(12) Patent Application Publication
(10) Pub. No.: US 2019/0167860 A1
(43) Pub. Date: Jun. 6, 2019

(54) METHOD FOR PRODUCING A PROTEIN-FUNCTIONALIZED FILM AS WELL AS PROTEIN FUNCTIONALIZED FILM

(71) Applicant: FRAUNHOFER-GESELLSCHAFT ZUR FÖRDERUNG DER ANGEWANDTEN FORSCHUNG E. V., München (DE)

(72) Inventors: Stefan REINICKE, Potsdam (DE); Alexander BÖKER, Berlin (DE)

(73) Assignee: FRAUNHOFER-GESELLSCHAFT ZUR FÖRDERUNG DER ANGEWANDTEN FORSCHUNG E. V., München (DE)

(21) Appl. No.: 16/324,105

(22) PCT Filed: May 24, 2017
(86) PCT No.: PCT/EP2017/062661

§ 371 Date: Feb. 7, 2019

(30) Foreign Application Priority Data
Aug. 12, 2016 (EP) 16184020.2

(51) Int. Cl. A61L 31/16; B05D 1/28; A61L 31/04; A61L 31/08

(52) U.S. Cl. CPC A61L 31/16; A61L 31/08; A61L 31/043; B05D 1/28

(57) ABSTRACT

The present invention relates to a method for producing a protein-functionalized film, in which a protein is bound to a copolymer or a polymer having an unhydrolyzed or hydrolyzed thiolactone functionalization is bound to the film by means of the existing functionalization. The present invention also relates to a correspondingly produced film.

The present invention relates to a method of producing a protein-functionalized film in which a protein is bonded to the film to a copolymer or a polymer having an unhydrolyzed or hydrolyzed thiolactone functionalization is bonded to the film via the existing functionalization. The present invention also relates to a correspondingly produced film.

The production of thin polymer films containing enzymes has been described a number of times and serves inter alia for the stabilization of the enzyme used and for the coupling of enzymatic reactions having suitable signal transmission paths. The small thickness of such films is above all interesting for areas of application in which only small quantities of active material are required, but in which at the same time fast response times or contact times are required that are not impeded by diffusion effects. Above all sensor systems, but also areas such as biocatalysis must be named here. However, no system has yet been found in which a single functional group takes over such a plurality of functions as part of the generation of the film containing enzymes.

The reversible bonding of enzymes to a polymer film via disulfide bridges has been described in a patent of 1979 (patent U.S. Pat. No. 4,176,006); however, the thiol groups here have to be introduced into the polymer film in a separate step via a low molecular mercaptan.

Compounds that are suitable for producing thin polymer films containing enzymes have to satisfy a whole series of properties. They must be miscible with aqueous enzyme solutions or must be swellable therein to achieve a sufficient charge with the enzyme. They have to bear functional groups that permit a firm bonding of the enzyme to the polymer film or of the polymer film to the substrate (e.g. covalently). A swelling ability of the finished polymer film in the working medium of the enzyme (aqueous solutions as a rule) is required and possibilities of cross-linking the polymer matrix are often aimed for. There is additionally above all the necessity of designing the immobilization process as protein compatible from the first to the last step, i.e. of largely preventing a deactivation of the enzyme during the process.

The production of ultrathin films by self-assembly procedures of suitable compounds (amphiphile molecules, polar polymers, and similar) at the air-water interface by means of the Langmuir technique has special importance since it offers the possibility of compressing thin films spread over the water surface in a controlled manner and to a desired degree and thus to set film thicknesses and film densities. Polymers as the material used have the advantage over low molecular amphiphile compounds here that they form mechanically more stable films. Usable polymers, however, have to have a limited water solubility and to have a certain polarity gradient structurally. This is typically solved by the use of amphiphile block copolymer structures that are, however, more complicated in manufacture than, for instance, homopolymers or statistical copolymers. In general, homopolymers or statistical copolymers can also form Langmuir films (there would be a polarity gradient along the structure of a repeat unit here), but the limited solubility would also have to be ensured here. Since, however, this contradicts the water swelling capability of the polymer matrix required for the later application, a way additionally has to be found to subsequently hydrophilize them.

The technical problem is therefore to find a polymer structure that has as simple a structure as possible, that is thus easily accessible, and that can take over a plurality of functions in the course of the formation of ultrathin films containing enyzmes on a solid substrate or at the air-water interface by means of the Langmuir technique: Hydrophobization of the polymer with a subsequent possibility of hydrophilization, covalent bonding of the enzyme to the polymer matrix, covalent bonding of the polymer matrix to the desired substrate, subsequent cross-linking of the film formed.

A method that uses a simple, easily accessible polymer structure that combines all the functions addressed above and that can also be acquired in part from biogenic raw materials does not yet exist. Functional groups in the polymers or polymer precursors as a rule each only take over one function. As a rule functional units such as oxirane groups, carboxyl groups, or amino groups are used for the covalent bonding of enzymes, with e.g. with the latter additional linkers or co-components, that may be toxic, being required to bond the enzyme. The enzyme is also frequently only physically embedded in a polymer matrix without a covalent bond. The cross-linking of the polymer matrix also typically takes place via cross-linkers that have to be additionally used.

To produce films on a polymer base at the air-water interface, amphiphile block structures are typically relied on, with the enzyme then as a rule only being physisorbed, but not covalently bonded. Any functional groups present also as a rule only take over one function in these cases.

Starting from this, it is thus the object of the present invention to provide a method of producing a protein-functionalized film that avoids the disadvantages addressed above. It is also the object of the present invention to provide a protein-functionalized film.

This object is achieved with respect to a method by the features of claim 1 and with respect to a protein-functionalized film by the features of claim 15. The respective dependent claims in this respect represent advantageous further developments.

The invention accordingly relates to a method of producing a protein-functionalized film in which a film containing at least one copolymer or a polymer having an unhydrolyzed and/or hydrolyzed thiolactone functionalization and at least one protein is produced or provided and the at least one protein is bonded covalently bonded to the at least one copolymer or polymer via the unhydrolyzed or hydrolyzed thiolactone functionalization.

It is the characterizing feature of the method in accordance with the invention that a specific copolymer or polymer is used that has an (unhydrolyzed or hydrolyzed) thiolactone functionalization. The thiolactone functionalization is here preferably worked into the copolymer or polymer via a corresponding monomer. The thiolactone functionalization can, however, also be present in a side chain of the copolymer or polymer, for example in the event the copolymers or polymers are graft polymers. Only the presence of the thiolactone functionalization is material to the invention for the covalent bonding of the protein.

The copolymer and/or the polymer can here comprise either only unhydrolyzed thiolactone functionalizations or only hydrolyzed thiolactone functionalizations. It is equally possible that the copolymer or the polymer has both kinds of functionalization.

The invention is in particular characterized by the following advantages:
A functional group—that satisfies up to 5 functions:
   covalent bonding of the polymer film to the substrate
   covalent bonding of the protein, in particular of an enzyme to the polymer film
   cross-linking of the polymer film
   control of the hydrophilia/hydrophobicity of the polymer
   provision of thiol groups for a reversible bonding of enzymes via a formation of disulfide bridges or for fixing the polymer matrix on metal surfaces
easy accessibility of this functional group from bio-based raw material sources
use of an easily accessible statistical copolymer produced by radical polymerization, avoidance of complicated amphiphile block structures or multifunctional structures.

The main advantage is that a functional group that is easily accessible and that can be just as easily installed in a polymer chain takes over a plurality of functions that are of importance in the course of the formation of thin films containing enzymes on different substrates. The use of complex multifunctional polymer structures or multcomponent systems is hereby avoided. At the same time, this functional group is a compound that is acquired e.g from bio-based raw material sources. The possibility of installing the thiolactone group in the most varied polymers furthermore leads to a comparatively flexible immobilization platform. Even though the method ultimately has to be individually tested for each enzyme, it can generally be applied to every protein that e.g. bears at least one lysin unit accessible from the outside.

The film of the copolymers or polymers can here be produced in situ in that the film is, for example, deposited from a solution of the copolymer or polymer. Alternatively, the film of the thiolactone-functionalized copolymer or polymer can also already be prepared in advance and can be provided for the purposes of the method in accordance with the invention.

In accordance with a preferred embodiment, the film is, however, produced in situ and is here in particular directly produced on a substrate or by means of the Langmuir-Schaefer technique.

A preferred embodiment of the production of the film on a substrate here comprises the coating with a doctor knife, spin coating and/or spray application from a solution of the at least one copolymer and/or polymer, preferably from a (weakly) acid or neutral solution, in particular from a solution having a pH of 5 to 7. Solvents are here used to produce the solutions of the copolymers and/or polymers that are able to dissolve the respective copolymers or polymers.

A preferred embodiment for producing the film by means of the Langmuir-Schaefer technique provides a spreading of a solution of the at least one copolymer and/or polymer in a volatile solvent that is not miscible with water, in particular chloroform, on the surface of water or of an aqueous solution.

For the case that the film of the copolymer or polymer is deposited on a substrate, the substrate is preferably selected from the group comprising polymer films or polymer membranes, preferably polymer films or membranes surface-functionalized with amino groups, in particular films or membranes surface-functionalized with amino groups and made of polyacrylonitrile, polydimethylsiloxane, polymers on a cellulose base, polyvinyl alcohols, poly(hydroxyalkyl acrylates), in particular poly(2-hydroxyethyl acetate), poly(hydroxymethyl acrylate), and copolymers thereof, and inorganic substrates, preferably inorganic substrates surface-functionalized with amino groups, in particular inorganic silicon wafers, glass substrates, or metal substrates surface-functionalized with amino groups, in particular coin metal substrates or substrates coated with coin metals. Gold is here particularly preferred as the coin metal. Metals that can enter into a covalent bond with thiol groups, for example the previously named coin metals, in particular gold, here do not necessarily have to be surface functionalized with amino groups.

A covalent bonding of the film to a substrate that may be present can thus take place via the existing thiolactone units in the at least one copolymer. For the case that the substrate has a superficially present amino functionalization, these amino groups can react with the thiolactone groups in an analogous manner to the amino groups of the protein to be bonded. On the other hand, alternatively or additionally to this, a covalent bonding of the copolymer or polymer to the substrate can take place directly via free SH groups of the hydrolyzed thiolactone unit, for example in the case of substrates composed of coin metals or of substrates coated with coin metals.

The protein can be brought into contact with the copolymers or polymers in various manners before production of the covalent bond and can thereby be worked into the film of the at least one copolymer or polymer.

The protein can, for example, be applied to the surface of the film, in particular by spin coating and/or spray application of a solution of the at least one protein after the production or provision of the film of the at least one copolymer or polymer. The film of the at least one copolymer or polymer is here penetrated by the at least one protein, i.e. the protein penetrates into the film of the copolymer or polymer.

Alternatively or additionally to this, it is equally possible that the at least one protein is mixed with the at least one copolymer and/or polymer and the film of the at least one copolymer and/or polymer is produced from this mixture. For example, the copolymer and/or polymer can be brought together with the at least one protein into a solution and the film can be produced from this solution. The at least one protein is in particular homogeneously distributed within the film of at least one copolymer or polymer in this embodiment.

In the case of the production of the film of the at least one copolymer and/or polymer at the air-water interface, the at least one protein can be worked in by means of the Langmuir-Schaefer technique by adding the protein into the aqueous subphase before, during and/or after the production of the film of the at least one copolymer and/or polymer and the adsorption and/or absorption of the at least one protein on and/or in the film of the copolymer and/or polymer.

Preferred unhydrolyzed thiolactone functionalizations are here in particular selected from the group of residues having the general formula I shown below

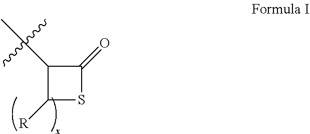

A hydrolyzed thiolactone functionalization is here in particular selected from the group comprising residues having the general formula II shown below

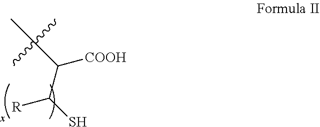

In the above-described formulas I and II, the following symbols mean, respectively independently of one another

R hydrogen or a linear or branched alkyl residue having 1 to 8 carbon atoms; and
x 1 to 6.

In accordance with a preferred embodiment, provision is made that the residue R means hydrogen and x is 2 or 3, in particular 2.

A further preferred embodiment provides that the copolymer contains repeat units or that the polymer is formed from repeat units having the following general formula III (unhydrolyzed) or IV (hydrolyzed)

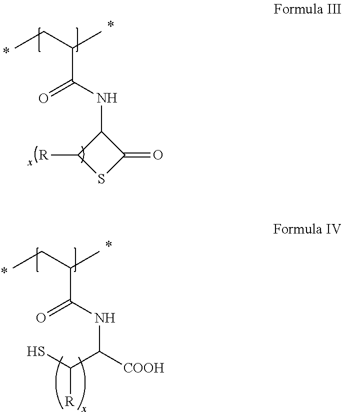

with the symbols meaning, respectively independently of one another
R hydrogen or a linear or branched alkyl residue having 1 to 8 carbon atoms; and
x 1 to 6
and preferably R being hydrogen and x being 2 or 3.

The copolymer can here additionally include repeat units that are not thiolactone-functionalized and in particular satisfy the general formula shown below:

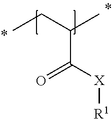

with the symbols meaning, respectively independently of one another

### X NH, 0 or NR¹ and

R¹ a linear or branched alkyl residue having 1 to 8 carbon atoms or hydrogen.

R¹ is here in particular an isopropyl residue and X is in particular the functionalization NH.

In accordance with a particularly preferred embodiment, the copolymer is formed from the following two repeat units a) and b):

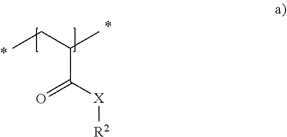

with the symbols meaning, respectively independently of one another

### X NH;

R² one of the following residues

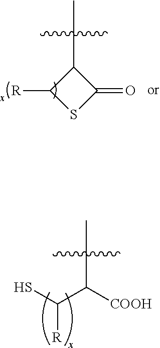

with the symbols meaning, respectively independently of one another
R hydrogen or a linear or branched alkyl residue having 1 to 8 carbon atoms; and
x 1 to 6;
and

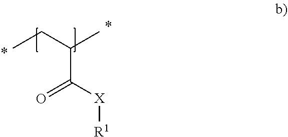

with the symbols meaning, respectively independently of one another
   X NH, 0 or NR¹ and
   R¹ a linear or branched alkyl residue having 1 to 8 carbon atoms or hydrogen,
      with the repeat units a) and b) being present in statistically distributed form in the copolymer.

It is in particular advantageous with the previously named preferred copolymer if the molar portion of the repeat units a) amounts, with respect to the totality of the repeat units a)+b) to 5 to 50 mol %, preferably 10 to 40 mol %, and particularly preferably 15 to 30 mol %.

The protein that is covalently bonded to the copolymer or polymer is preferably an enzyme, in particular an enzyme selected from the group comprising aldolases, hydrolases, such as lipases, proteases, amidases, acylases, nitrilases, dehalogenases, isomerases, transferases, or a functional protein, in particular channel proteins and antibodies, and combinations thereof.

The film can, for example, be charged with 0.01 to 50 wt %, preferably 1 to 20 wt %, of the at least one protein.

A further preferred embodiment provides that the film containing protein produced in the first step is stored over 0.1 to 24 h, preferably 0.1 to 8 h, in particular 0.1 to 2 h, for the covalent bonding of the protein to the at least one copolymer or polymer having an unhydrolyzed thiolactone functionalization.

The previously described storage is here carried out with at least one of the following parameters: At temperatures of 0 to 30° C., preferably 0 to 10° C., and/or at a pH of 7.5 to 12, preferably 8 to 10, and/or under the effect of an oxidation agent, hydrogen peroxide, for example.

A cross-linking of the at least one copolymer and/or polymer in particular takes place in the previously named post-treatment steps in that still present thiolactone functionalities are hydrolyzed and existing free thiol functionalities and thiol functionalities created by hydrolysis form disulfide bridges with one another.

In addition, a covalent linking of the at least one copolymer and/or polymer to a substrate that may be present is in particular possible via the described post-treatment in that, for example, an amino group of a substrate superficially amino group-functionalized reacts with a still present thiolactone or a free thiol group reacts with a substrate affine with a thiol group, preferably a metal or a substrate coated with a metal, in particular a coin metal or a substrate coated with a coin metal, for example a gold substrate or a substrate coated with gold while forming a covalent bond.

Alternatively to this, it is equally preferred that the film containing a protein produced in the first step is treated under oxidizing conditions for the covalent bonding of the protein to the at least one copolymer or polymer having a hydrolyzed thiolactone functionalization, with disulfide bridges being produced between the protein's own thiol groups and the thiol groups of the hydrolyzed thiolactone.

The invention additionally relates to a protein-functionalized film, containing at least one copolymer and/or polymer, to which at least one protein is covalently bonded via a spacer selected from the general formulas V and VI shown below

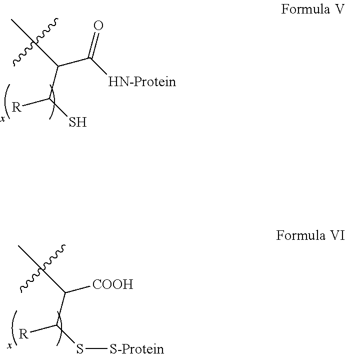

with the symbols meaning, respectively independently of one another
R hydrogen or a linear or branched alkyl residue having 1 to 8 carbon atoms; and
x 1 to 6.

The free SH groups in formula V can form, in part or in total, disulfide bridges to further free SH groups of formula V and/or to thiol groups of proteins by the post-treatment step and can thus stabilize the polymer film in a cross-linking manner.

In addition, a covalent bonding of the substrate can also take place via the thiolactone functionalization in accordance with the mechanisms further above.

All the previously named preferred embodiments that are named in connection with the method, in particular with respect to the usable or bonded proteins, the preferred embodiments with respect to the functional groupings used for the bonding, and possible substrates, equally apply without restriction to the protein-functionalized film in accordance with the invention.

The protein-functionalized film in accordance with the invention is in particular characterized by a thickness of 5 nm to 500 nm.

The film can here, as described above, equally be arranged on a substrate that has been previously already been defined in detail.

A further preferred embodiment provides that the content of the at least one protein amounts, with respect to the total mass of the film, to from 0.01 to 50 wt %, preferably 1 to 20 wt %.

Technical application possibilities of the present invention are in particular the immobilization of enzymes on membrane substrates for biocatalytic applications. It is here above all the possibility of increasing the enzyme stability, of avoiding laborious cleaning steps, and of permitting continuous process management. The immobilization described in more detail above by way of example of an aldolase, an enzyme that is used in the manufacture of active pharmaceutical ingredients, can be named as an example here. Established, scalable membrane drawing methods can in principle be used for the manufacture of such membranes. The possibility of regenerating the enzyme-active membrane, i.e. of recharging it with an enzyme without the necessity of having to completely replace the membrane, may well represent an important plus factor for a number of technical applications. A further relevant area of application could be found in sensor systems. Glucose sensors, for example, are often based on glucose oxidase that is immobilized in thin films.

The present invention will be looked at in more detail with reference to the following embodiments without restricting the invention to the specifically shown parameters.

The subject matter of the present invention will be explained in more detail in the following, in particular for the example of a use of copolymers containing thiolactone for different methods for producing thin polymer films or layers containing enzymes on varying substrates.

These films or layers can here either be generated directly on the desired substrate by techniques such as spin coating and spray application or can first be formed at the air-water interface with a subsequent transfer to the substrate by means of the Langmuir-Schaefer technique. The central element here is formed by the γbutyrothiol actone units of the copolymer used that are preferably arranged along the polymer chain as statistically distributed substituents.

They adopt essential functions for the respective immobilization processes whose combination can typically only be achieved by use of structurally complex compounds.

At the same time, γbutyrothiol actone derivatives can be easily acquired from biological raw material sources as products of the intramolecular condensation reaction of methionine or homocysteine.

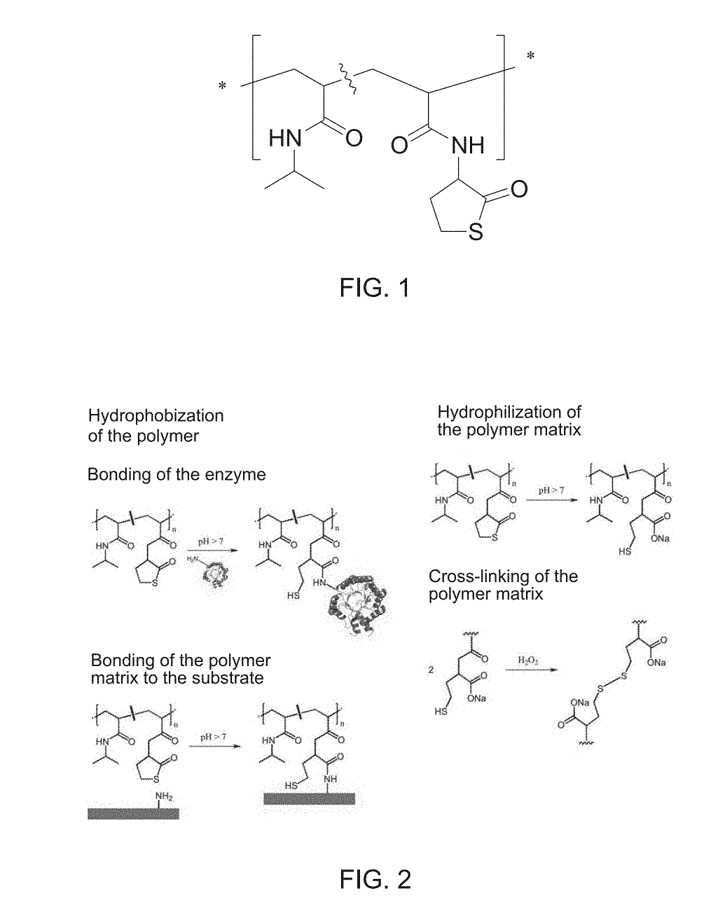

The production of the thin polymer film containing an enzyme preferably takes place in two stages. First, the film itself is generated on a suitable substrate, with the enzyme here first being physically integrated into the polymer layer. In a post-treatment step, the firm bonding of all the components and the cross-linking and, under certain circumstances, the hydrophilization, of the polymer matrix is then ensured. Different polymer classes can be considered for the basic structure of the polymer used such as systems based on (meth)acrylamide or (meth)acrylate. They can be easily synthesized by radical copolymerization of the main monomer with an analog, thiolactone-functionalized comonomer. If N-isopropyl acrylamide is assumed as the main monomer unit (see FIG. 1 in which the basic structure of a statistical copolymer containing thiolactone on a basis of N-isopropyl acrylamide is shown), a polymer is obtained by a sufficiently high portion of a thiolactone acrylamide comonomer that is able to form defined Langmuir films and is sufficiently hydrophobic here so that this film remains stable at the air-water interface over a longer time period. If, on the other hand, N—N-dimethylacrylamide is used as the main monomer unit, polymers can in turn be produced that are also still soluble in water with higher thiolactone contents, which is above all important for the method via the direct application of the film onto a substrate.

All the functions that the thiolactone units adopt in the course of the immobilization method are shown in FIG. 2. The functions taken over by the thiolactone groups of the polymer used in the course of the generation of thin polymer films containing enzymes on varying substrates can be seen.

### A) Generation of the Thin Film Containing an Enzyme

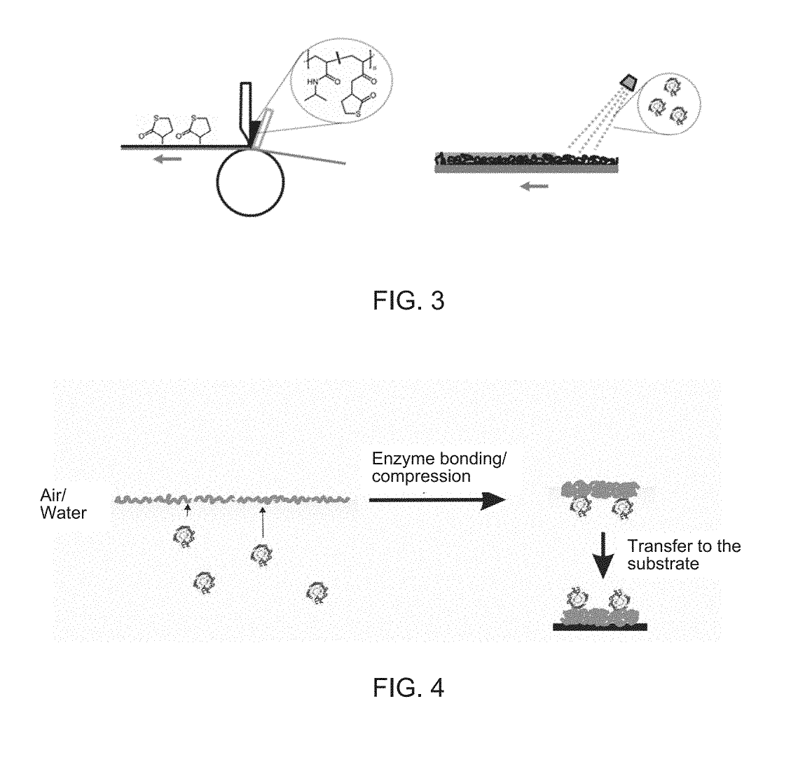

The direct application of the polymer film onto a substrate, for example a polyacrylonitrile membrane, that is surface-functionalized with amino groups, can take place by coating with a doctor knife, spin coating, or spray application, from a slightly acidic solution. The charge with the enzyme then likewise takes place by spray application. (FIG. 3). The application of the polymer film by coating with a doctor knife is shown at the left in FIG. 3; the charging of the applied polymer film with an enzyme solution by spray application is shown at the right.

Alternatively, the polymer and the enzyme are mixed in a solution and then applied together.

In the case of the film generation at the air-water interface by means of Langmuir Schaefer, the polymer film is generated in that a solution of the polymer in chloroform is spread on the water surface. After the evaporation of the solvent, the film can be compressed as desired by movement of the barriers of the Langmuir trough before the protein to be immobilized, an aldolase by way of example here, is subsequently injected into the subphase. After a fixed waiting period (approximately 1 to 2 hours) during which the enzyme is adsorbed at the polymer film, said film is transferred after a possible further compression and thus compaction by a simple bouncing of a suitable amine-functionalized substrate on the water surface to that substrate (see FIG. 4 that schematically shows the generation of an ultrathin polymer film containing an enzyme by means of the Langmuir-Schaefer technique). An ultrathin film having a desired film thickness can be built up in a controlled manner by a multiple repetition of the transfer step (see FIG. 4). The controlled buildup of an ultrathin film of a polythiolactone on an N-isopropylacrylamide (NIPAAm) base from FIG. 1 on a suitable substrate is shown in this respect (here: amino-functionalized silicon wafers) by means of Langmuir-Schaefer; A) Film thickness in dependence on the number of transfer steps; B) AFM topography image of a polymer film created from consecutive 8 transfer steps.

### B) Post-Treatment Step

The post-treatment of a polymer film containing aldolase and produced by Langmuir-Schaefer will be described in the following. The post-treatment of films that are acquired by direct application, however, takes place in a completely analog manner. Subsequent to the film generation and film transfer, the coated substrate is stored in a slightly basic buffer (pH=9) at 5° C. for some hours to force the bonding of the enzyme to the polymer and the fixing of the polymer to the substrate via the thiolactone units (see also FIG. 2 in this respect). At the same time, a further portion of the thiolactones is hydrolyzed, whereby the polymer matrix is hydrophilized. The conversion of the thiolactones in turn causes the release of thiol groups that can form disfulfide bridges under oxidative conditions and thus effect an additional cross-linking of the polymer film. To ensure the latter to a sufficient degree, small amounts of hydrogen peroxide are added. The successful hydrophilization can be demonstrated by contact angle measurements and AFM studies. If no correspondingly amine-functionalized material is available for the bonding of the polymer matrix to the substrate, it can also take place in principle via the released thiol groups. This bonding strategy can, for example, be considered for metal surfaces.

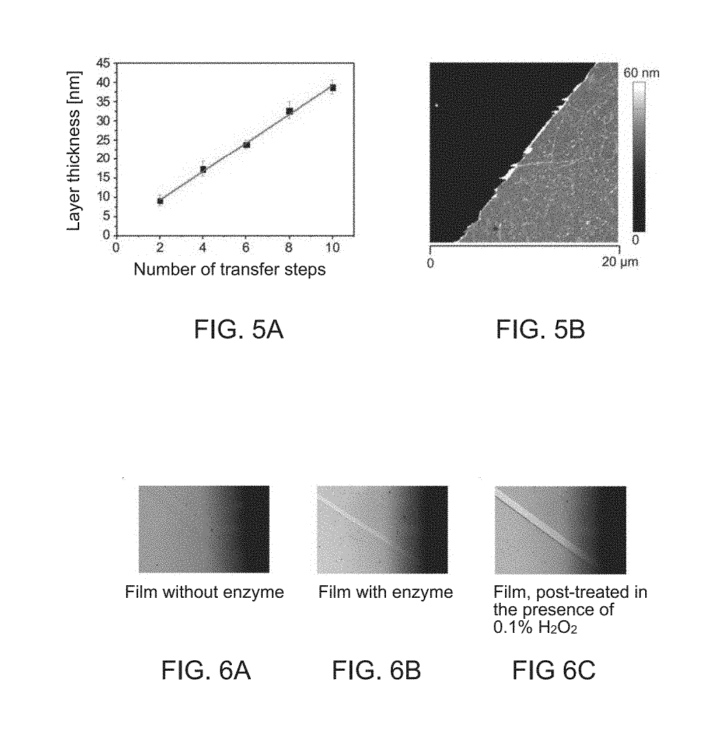

It was able to be shown by the use of enzymes marked in advance by a fluorescent dye that the films generated at the air-water interface actually do contain enzymes, with the degree of the initial compression of the film directly influencing the immobilized amount of enzyme. Since the enzyme used like most proteins represents a multifunctional amine, its bonding to the thiolactone units of the polymer matrix already causes a cross-linking thereof. This becomes clear with reference to microscope images that show that the post-treatment of the transferred films leaves them a lot more intact if an enzyme is present while a large part of the material is washed off during the post-treatment in the absence of enzyme (see FIG. 6 in this respect in which optical microscope images of thin polythiolactone films on an NIPAAm base on silicon wafers are shown that are generated by means of Langmuir-Schaefer after the treatment with a weakly basic potassium phosphate buffer (pH=9)). The contrast between the regions with a film and those without a film correlates directly with the film thickness and thus with the amount of material that is left over after the post-treatment step. In general, however, more material remains if H₂O₂ is used to form the disulfide bridges.

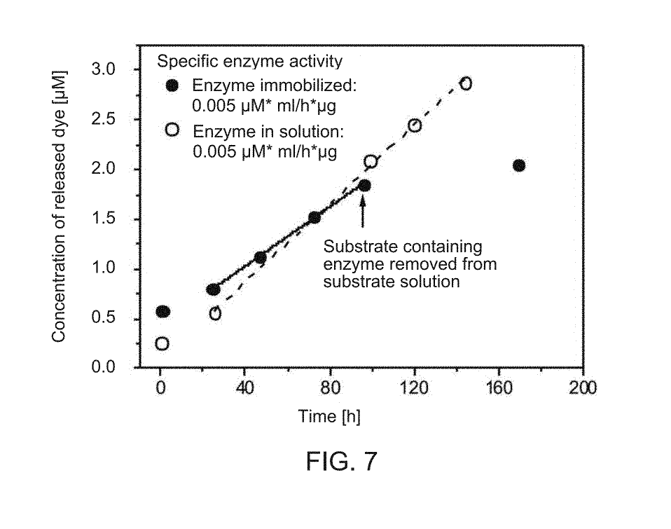

The transferred films show enzymatic activity (FIG. 7), with an out-diffusion of the enzyme not being able to be observed or with only a small portion being able to be observed in the course of the measurements. An activity assay for 2-deoxy-5-phosphate aldolase (DERA) based on the release of a fluorescent dye in the course of the enzymatic conversion of a corresponding substrate is shown in FIG. 7. The respective concentration of enzyme (immobilized and in solution) amounted to approximately to 3 μg/mL (with respect to the volume of the substrate solution). A conclusion can be drawn from the results of the activity studies with the aid of fluorescent measurements with which the respective quantity of immobilized enzyme can be estimated that the specific activity of the immobilized enzyme and that of the corresponding enzyme in solution are of a similar order of magnitude. A huge loss of enzyme activity is effectively avoided by the immobilization strategy in accordance with the invention.

### C) Modified Protocol for the Production of Regenerable Membranes

A slight modification of the method for the direct application of the polymer film containing an enzyme onto a substrate provides the possibility of building up regenerable films containing enzymes. The polymer is here first applied to a substrate on its own in the manner described under A). Before the charge with enzyme takes place, however, a hydrolysis of the thiolactone units is now first carried out. It is important here that the hydrolysis is complete so that later no enzyme can irreversibly bond to the polymer film in accordance with FIG. 3. The bonding instead takes place by generation of disulfide bridges between the enzyme's own thiol groups and the polymer's own thiol groups released in the hydrolysis step under oxidative conditions. These bonds can be split again at a later time by a suitable reductant in order thus to make the way free for a repeat charge with enzyme. The thiolactone units here also continue to serve the bonding of the polymer film to the substrate.

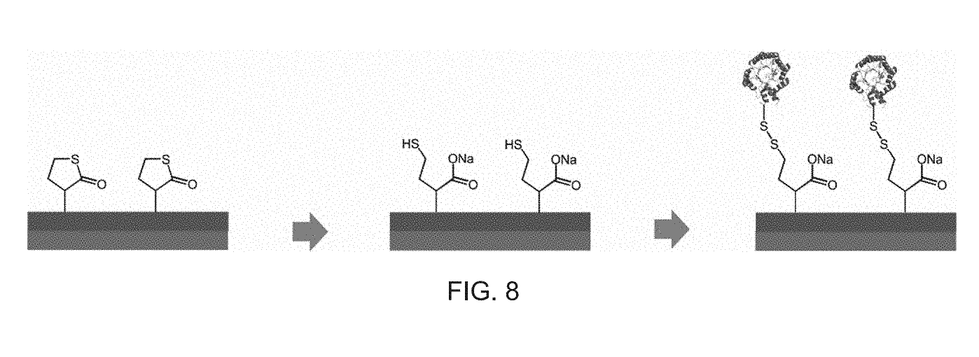

A corresponding procedure is outlined in FIG. 8 that schematically shows a method of producing regenerable polymer films containing enzymes by reversible bonding of the enzyme to the polymer matrix via a formation of disulfide bridges.

1-15. (canceled)

16. A method of producing a protein-functionalized film in which a film comprising
at least one copolymer or polymer having an unhydrolyzed and/or hydrolyzed thiolactone functionalization and at least one protein are produced or provided, and
the at least one protein is covalently bonded to the at least one copolymer or polymer via the unhydrolyzed or hydrolyzed thiolactone functionalization.

17. The method in accordance with claim 16, wherein the protein-functionalized film is produced directly on a substrate or by means of a Langmuir-Schaefer technique.

18. The method in accordance with claim 17, wherein the production of the protein-functionalized film takes place
on a substrate by coating with a doctor knife, spin coating and/or spray application from a solution of the at least one copolymer and/or polymer, and/or
by means of a Langmuir-Schaefer technique by spreading a solution of the at least one copolymer and/or polymer in a volatile solvent not miscible with water over the surface of water or of an aqueous solution (aqueous subphase).

19. The method in accordance with claim 18, wherein the substrate is selected from the group of polymer films and polymer membranes.

20. The method in accordance with claim 16, wherein the at least one protein
is applied to the surface of the film; and/or
is worked into the film of the copolymer and/or polymer during the production of the film by mixing the at least one protein with the at least one copolymer and/or polymer; and/or
in the case of the production of the film of the at least one copolymer and/or polymer by means of the Langmuir-Schaefer technique by adding the protein into the aqueous subphase before, during and/or after the production of the film of the at least one copolymer and/or polymer and the adsorption and/or absorption of the at least one protein on and/or in the film.

21. The method in accordance with claim 16, wherein the unhydrolyzed thiolactone functionalization is selected from the group consisting of residues having the general formula I shown below and the hydrolyzed thiolactone functionalization is selected from the group of residues having the general formula II shown below 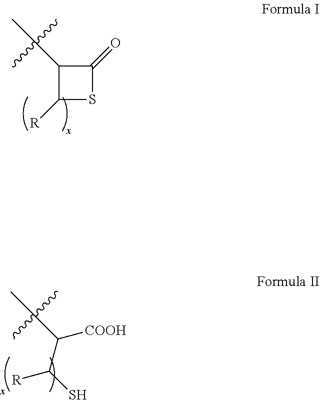
with the symbols meaning, respectively independently of one another
R hydrogen or a linear or branched alkyl residue having 1 to 8 carbon atoms; and
x 1 to 6.

22. The method in accordance with claim 16, wherein the copolymer contains repeat units or that polymer is formed from repeat units having the following general formula III (unhydrolyzed) or IV (hydrolyzed) 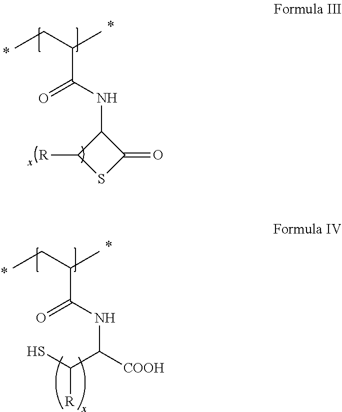
with the symbols meaning, respectively independently of one another
R hydrogen or a linear or branched alkyl residue having 1 to 8 carbon atoms; and
x 1 to 6.

23. The method in accordance with claim 16, wherein the copolymer contains repeat units of the general formula shown below 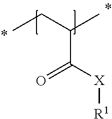
with the symbols meaning, respectively independently of one another
X NH, O or NR¹ and
R¹ a linear or branched alkyl residue having 1 to 8 carbon atoms or hydrogen.

24. The method in accordance with claim 16, wherein the copolymer is formed from the two following repeat units a) and b): 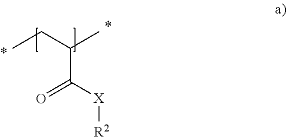
with the symbols meaning, respectively independently of one another
X NH;
R² one of the following residues 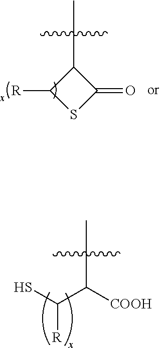
R hydrogen or a linear or branched alkyl residue having 1 to 8 carbon atoms; and
x 1 to 6;
and 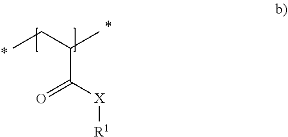
with the symbols meaning, respectively independently of one another
X NH, 0 or NR′ and
R¹ a linear or branched alkyl residue having 1 to 8 carbon atoms or hydrogen,
with the repeat units a) and b) being present in statistically distributed form in the copolymer.

25. The method in accordance with claim 16, wherein the protein is an enzyme.

26. The method in accordance with claim 16, wherein the film is charged with 0.01 to 50 wt % of the at least one protein.

27. The method in accordance with claim 16, wherein the film containing protein produced in the first step is stored over 0.1 to 24 h for the covalent bonding of the protein to the at least one copolymer or polymer having an unhydrolyzed thiolactone functionalization.

28. The method in accordance with claim 27, wherein the storage takes place
at temperatures from 0 to 30° C.; and/or
at a pH of 7.5 to 12; and/or
under the effect of an oxidation agent.

29. The method in accordance with claim 16, wherein the film containing a protein produced in the first step is treated under oxidizing conditions for the covalent bonding of the protein to the at least one copolymer or polymer having a hydrolyzed thiolactone functionalization, with disulfide bridges being produced between the protein's own thiol groups and the thiol groups of the hydrolyzed thiolactone.

30. A protein-functionalized film, containing at least one copolymer and/or polymer, to which at least one protein is covalently bonded via a spacer selected from the general formulas V and VI shown below 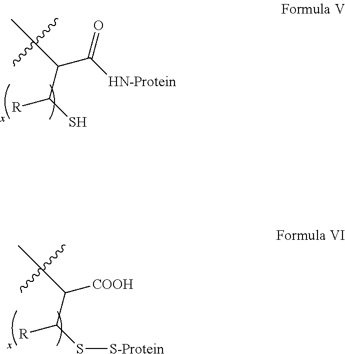
with the symbols meaning, respectively independently of one another
R hydrogen or a linear or branched alkyl residue having 1 to 8 carbon atoms; and
x 1 to 6.
